(19) United States
(12) Patent Application Publication
(10) Pub. No.: US 2014/0068798 A1
(43) Pub. Date: Mar. 6, 2014

(54) Multi-Seed Mutant of Sorghum for Increasing Grain Yield

(71) Applicants: Zhanguo Xin, Lubbock, TX (US); Gloria B. Burow, Lubbock, TX (US); John J. Burke, Lubbock, TX (US)

(72) Inventors: Zhanguo Xin, Lubbock, TX (US); Gloria B. Burow, Lubbock, TX (US); John J. Burke, Lubbock, TX (US)

(21) Appl. No.: 13/599,183

(22) Filed: Aug. 30, 2012

(51) Int. Cl. A01H 5/00; C12N 5/04; A01H 1/02; A01H 5/10

(52) U.S. Cl. 800/260; 800/320; 435/410

(57) ABSTRACT

Stable and heritable sorghum mutants are produced in which the development arrest of the pedicellate spikelets is released. In these mutants, all spikelets, both sessile and pedicellate, develop into flowers and produce mature seeds, thereby significantly increasing seed production and yield in comparison to wild-type sorghum. These mutants may be crossed with other sorghum lines, particularly elite large-seeded lines, to improve grain yield in sorghum and other related species.

### BACKGROUND OF THE INVENTION

1. Field of the Invention

This invention relates to multi-seed sorghum mutants which develop seeds at not only the sessile spikelets of the panicles, but also at the pedicellate spikelets, thereby significantly increasing the seed production and yield.

2. Description of the Prior Art

Seed number per panicle is a major determinant of grain yield in sorghum [Sorghum bicolor (L.) Moech] and other crops (Saeed et al. 1986. Yield Component Analysis in Grain Sorghum. Crop Sci. 26:346-351; Duggan et al. 2000. Yield component variation in winter wheat grown under drought stress. Can. J. Plant Sci. 80:739-745; Richards. 2000. Selectable traits to increase crop photosynthesis and yield of grain crops. J. Exp. Bot. 51:447-458; Ashikari et al. 2005. Cytokinin Oxidase Regulates Rice Grain Production. Science 309:741-745; Reynolds et al. 2009. Raising yield potential in wheat. J. Exp. Bot. 60:1899-1918). Increased seed number and seed size, which directly are related to high grain yield, is a common goal during domestication of cereal crops resulting in inadvertent selection of genetic stocks with greater number of and larger seeds (Zohary et al. 2012. Domestication of Plants in the Old World: The Origin and Spread of Cultivated Plants in West Asia, Europe, and the Mediterranean Basin. 4 ed. Oxford University Press, Oxford, U.K). The number of seeds per panicle in sorghum is determined primarily by conserved inflorescence architecture and panicle morphology. Sorghum inflorescence consists of a main rachis on which many primary branches are developed. Secondary branches, sometimes, tertiary branches are developed from the primary branch (Brown et al. 2006. Inheritance of inflorescence architecture in sorghum. Theor. Appl. Genet. 113:931-942). The main inflorescence, primary branches, secondary, and tertiary branches, all end with a terminal spike, which consists one sessile complete spikelet (floret) and two sterile pedicellate spikelets (florets with a pedicel) (Walters and Keil. 1988. Vascular Plant Taxonomy. 4th ed. Kendall/Hunt Pub. Co., Dubuque, Iowa). Below the terminal spike, one or more spikes can develop (FIG. 1A). These adjacent spikes usually consist of one sessile and one pedicellate spikelet. The sessile spikelets of terminal or adjacent spikes are complete flowers that will develop into seeds, while the pedicellate spikelets develop only sterile flower structures and result in chaffy structures. In some sorghum lines, the pedicellate spikelets can develop one to three anthers but completely lack gynoecial organs.

Mutagenesis by radiation or chemical mutagens is an effective approach to elucidate morphogenesis, metabolism, and signal transduction pathways in both prokaryote and eukaryote organisms, including higher plants (Bentley et al. 2000. Targeted recovery of mutations in Drosophila. Genetics 156:1169-73; Henikoff et al. 2004. TILLING. Traditional mutagenesis meets functional genomics. Plant Physiol 135:630-6; Amsterdam and Hopkins. 2006. Mutagenesis strategies in zebrafish for identifying genes involved in development and disease. Trends Genet 22:473-8). It has also long been applied to sorghum to isolate novel phenotypes that may have potential application in breeding (Quinby and Karper. 1942. Inheritance of Mature Plant Characters In Sorghum: Induced by Radiation. J. Hered. 33:323-327; Gaul. 1964. Mutations in plant breeding. Rad. Bot. 4:155-232). Many mutants with unique phenotypes that have not been observed in natural sorghum collections have been identified from mutant populations treated with various mutagens, such as X-ray and γ-irradiation, ethyl methane sulfonate (EMS), methyl methane sulfonate (MMS), diethyl sulfate (DES), N-Nitroso methyl urea (NMU), N-Nitroso ethyl urea (NEU), or combinations of chemical and irradiation mutagens (Quinby and Karper. 1942. ibid; Sree Ramulu. 1970a. Induced systematic mutations in Sorghum. Mutation Research/Fundamental and Molecular Mechanisms of Mutagenesis 10:77-80; Sree Ramulu. 1970b. Sensitivity and induction of mutations in sorghum. Mutation Research/Fundamental and Molecular Mechanisms of Mutagenesis 10:197-206; Sree Ramulu and Sree Rangasamy. 1972. An estimation of the number of initials in grain Sorghum using mutagenic treatments. Rad. Bot. 12:37-43). Many beneficial mutations, including dwarfing, early flowering, high protein digestibility, high lysine and others, have been widely used in sorghum breeding (Singh and Axtell. 1973. High Lysine Mutant Gene (hl that Improves Protein Quality and Biological Value of Grain Sorghum. Crop Sci. 13:535-539; Quinby. 1975. The Genetics of Sorghum Improvement. J. Hered. 66:56-62; Ejeta and Axtell. 1985. Mutant gene in sorghum causing leaf “reddening” and increased protein concentration in the grain. J Hered 76:301-302; Oria et al. 2000. A highly digestible sorghum mutant cultivar exhibits a unique folded structure of endosperm protein bodies. Proc. Natl. Acad. Sci. USA 97:5065-70). The late Dr. Keith Schertz, a former sorghum geneticist with USDA-ARS, collected and preserved more than 507 natural and historic mutant lines from various genetic sources.

The completion of the genome sequence in a leading inbred line, BT×623, has made it possible to study gene function on a genome-wide scale, and to compare gene function with other plants (Paterson. 2008. Genomics of sorghum. International journal of plant genomics 2008:362451; Paterson et al. 2009. The Sorghum bicolor genome and the diversification of grasses. Nature 457:551-556).

However, despite these and other advances, the need remains for improved sorghum lines providing increased yield.

### SUMMARY OF THE INVENTION

We have now produced and isolated a novel class of stable (fertile) and heritable sorghum mutants in which the development arrest of reproductive structures of pedicellate spikelets is released. In these mutants, all spikelets of the sorghum plant, both sessile and pedicellate, develop into flowers and produce mature, viable seeds, thereby significantly increasing seed production and yield in comparison to wild-type sorghum. These mutants may be crossed with other sorghum lines, particularly elite large-seeded lines, to improve grain yield in sorghum and other related species.

In accordance with this discovery, it is an object of this invention to provide sorghum which produce mature seed at both the sessile and pedicellate spikelets.

Another object of this invention is to provide stable and heritable sorghum mutants with greater seed production and increased grain yield.

A further object of this invention is to provide stable and heritable sorghum mutants which produce mature seed at both the sessile and pedicellate spikelets, which mutants may be crossed with other sorghum lines to increase seed number and improve grain yield.

Other objects and advantages of this invention will become readily apparent from the ensuing description.

### BRIEF DESCRIPTION OF THE DRAWINGS

FIG. 1 is a diagram of sorghum panicles showing the basic structure of terminal and adjacent spikes. The terminal spike is composed of a sessile (middle) and two pedicellate accessory spikelets (represented as oval figures). Non-terminal or adjacent spikes are composed of a sessile and a single pedicellate accessory spikelet. In (A) wild-type BT×623 (MSD=mono seeded) spikes, only the sessile spikelet develops into seeds (shown as filled ovals) while pedicellate spikelets are composed of bracts that do not develop reproductive organs and become chaffy structures (shown as open ovals). In (B) multi-seeded (msd) mutant, all spikelets (sessile and pedicellate) of terminal and adjacent spikes produce reproductive organs and develop into seeds, thus all spikelets are shown as filled ovals.

FIG. 2 shows the characterization of various inflorescence attributes of WT and msd1. (A) Length of the primary branch of inflorescence; (B) Number of primary branches per panicle (**) and (C) Number of secondary branches per primary branch (*).

FIG. 3 shows the seed production of wild-type (WT) and msd1 mutants. (A) Number of seeds/primary branch (msd12 contained double the seed number of WT); (B) 100 seed weight (msd12 has lighter seeds than WT); (C) Total seed weight per panicle (grain yield).

### DEFINITIONS

Allele: the term coined by Bateson and Saunders (1902) for characters which are alternative to one another in Mendelian inheritance (Gk. Allelon, one another; morphe, form). Now the term allele is used for two or more alternative forms of a gene resulting in different gene products and thus different phenotypes. In a haploid set of chromosomes there is only one allele at its specific locus. Diploid organisms have 2 alleles at a given locus, and if they are homozygous for a defined gene, both alleles are identical. However, if heterozygous for a defined gene they have one normal and one mutant allele. A single allele for each gene locus is inherited separately from each parent (e.g., at a locus for eye color the allele might result in blue or brown eyes). An organism is homozygous for a gene if the alleles are identical, and heterozygous if they are different. (Birgid Schlindwein's Hypermedia Glossary of Genetic Terms).

Androecium: the collective term for all stamens of a flower or “male reproductive structures of a flower”. (Dirk R. Walters; David J. Keil's Vascular Plant Taxonomy 4^{th} edition; Walter S. Judd et al. Plant Systematics: A Phylogenetic Approach; Sinauer Assoc. Sunderland, Mass., USA).

Awns: bristles arising from a spikelet part. Some lines have a very small awn, called a tip awn. Awn presence is indicated as present or not present.

Backcrossing: a process in which a breeder repeatedly crosses hybrid progeny back to one of the parents. For example, a first generation hybrid F₁ may be crossed with one of the parental genotypes of the F₁ hybrid.

Chaff: dry scale like structures used collectively to describe dried out sterile bracts and structures as in some species of Poaceae and Asteraceae (Dirk R. Walters; David J. Keil's Vascular Plant Taxonomy 4^{th} edition).

DNA or RNA sequence: a linear series of nucleotides connected one to the other by phosphodiester bonds between the 3′ and 5′ carbons of adjacent pentoses.

Floret: in Poaceae, the unit composed of lemma and palea and the small flower they enclose or any small flower of dense inflorescence (Dirk R. Walters; David J. Keil's Vascular Plant Taxonomy 4^{th} edition).

Genotype: the term proposed by Johannsen (1909) for the hereditary constitution of an individual, or of particular nuclei within its cells. (Birgid Schlindwein's Hypermedia Glossary of Genetic Terms).

Glume Color: refers to one of a pair of empty scales at the base of a spikelet. Glume color is typically described as tan, mahogany, red, purple, or black.

Gynoecium: collective term for all carpels of the flower or “the female reproductive structures” (Dirk R. Walters; David J. Keil's Vascular Plant Taxonomy 4^{th} edition).

Immature Seed: in contrast to mature seed, immature seed lack a fully developed endosperm and/or embryo, and is incapable of germination and development into a mature plant.

Leaf Angle: refers to the angle between the leaves and the stalk.

Leaf Length and Width: is measured by selecting the largest leaf, after flowering, on a representative sample of plants and measuring the maximum length and width. Generally, this will be a leaf towards the middle of the plant.

Leaf Mid-Rib Color: can be described as white, cloudy, intermediate, or brown. White indicates a dry mid-rib and stalk, while cloudy indicates that they are juicy. Brown indicates the presence of a mutant allele that conditions for a reduced amount of lignin in the plant.

Leaf Number: is measured by counting the total number of leaves on the main stalk after flowering. Some of the first leaves may have deteriorated by that time, so an estimate can be made.

Locus: the position of a gene on a chromosome or other chromosome markers; also, the DNA at that position. The use of the term locus is sometimes restricted to main regions of DNA that are expressed. (Birgid Schlindwein's Hypermedia Glossary of Genetic Terms).

Maturity: the measurement of the number of days between planting and physiological maturity.

Mature Seed: seed having a fully developed, viable embryo, seed coat and endosperm, which seed is capable of germination and development into a mature plant.

Mutant: refers to any stable plant whose functional properties are different from the parent line.

Nucleic acid: a deoxyribonucleotide or ribonucleotide polymer in either single- or double-stranded form, including known analogs of natural nucleotides unless otherwise indicated.

Nucleotide: a monomeric unit of DNA or RNA consisting of a sugar moiety (pentose), a phosphate, and a nitrogenous heterocyclic base. The base is linked to the sugar moiety via the glycosidic carbon (1′ carbon of the pentose) and that combination of base and sugar is a nucleoside. The base characterizes the nucleotide. The four DNA bases are adenine (“A”), guanine (“G”), cytosine (“C”) and thymine (“T”). The four RNA bases are A, G, C and uracil (“U”).

Panicle: type of inflorescence with two or more orders of branching, each axis bearing flowers or higher axes (Dirk R. Walters; David J. Keil's Vascular Plant Taxonomy 4^{th} edition).

Panicle Branch Attitude: an indicator of the attitude of the panicle branch with reference to the stalk, where erect indicates a panicle branch angle (central rachis to panicle branch axil to panicle branch) less than about 45 degrees, semi-erect refers to a panicle branch angle of about 45-80 degrees, and horizontal refers to a panicle branch angle of about 80 degrees or greater.

Panicle Branch Length: measured by selecting panicle branches from the middle of the panicle, which are generally the longest, and measuring the length in inches.

Panicle Length: the length of the panicle from the attachment point of the lowest branch to the tip of the uppermost branch in its normal orientation.

Panicle (or Head) Type: an indicator of the morphology of a sorghum plant's head (panicle), where open indicates an open panicle characterized by either more distance between the panicle branches or longer panicle branches; semi-open indicates a less open panicle; semi-compact indicates a semi-compact panicle caused by shorter panicle branches arranged more closely on the central rachis; and compact indicates a very compact panicle caused by very short panicle branches arranged tightly on the central rachis.

Panicle Shape: an indicator of the shape of a sorghum plant's head (panicle) selected from cylindrical, elliptical, oval, and round.

Pedicel: the stalk of a spikelet in Poaceae or Cyperaceae (Dirk R. Walters; David J. Keil's Vascular Plant Taxonomy 4^{th} edition).

Pedicellate: refers to attachment of a spikelet through a stalk or pedicel in Poaceae.

Phenotype: the term coined by Johannsen (1909) for the appearance (Gk. phainein, to appear) of an organism with respect to a particular character or group of characters (physical, biochemical, and physiologic), as a result of the interaction of its genotype and its environment. Often used to define the consequences of a particular mutation. (Birgid Schlindwein's Hypermedia Glossary of Genetic Terms).

Plant Color: results from the presence or absence of anthocyanin pigments in the stalks and other organs of sorghum plants. The type and degree of coloration is determined by genotype and is somewhat subject to growing conditions, but varieties typically show varying degrees of coloration ranging from: absent (tan plant) to very strong (deep purple coloration). Ratings generally are tan, red, or purple.

Plant Height: the average height of the plant at the end of flowering, assuming the plant is not lodged. This varies from variety to variety and although it can be influenced by environment, relative comparisons between varieties grown side by side are useful for variety identification. Plant height is measured from the ground to the tip of the panicle.

Sessile: without a stalk; positioned directly against another structure, in sorghum the spikelets are directly attached to the rachis (Dirk R. Walters; David J. Keil's Vascular Plant Taxonomy 4^{th} edition) (Walter S. Judd et al. Plant Systematics: A Phylogenetic Approach; Sinauer Assoc. Sunderland, Mass., USA).

Spike: simple indeterminate inflorescence with single axis bearing sessile flowers (Walter S. Judd et al. Plant Systematics: A Phylogenetic Approach; Sinauer Assoc. Sunderland, Mass., USA).

Spikelet: basic inflorescence units of members of Poaceae and Cyperaceae (Walter S. Judd et al. Plant Systematics: A Phylogenetic Approach; Sinauer Assoc. Sunderland, Mass., USA).

Tillering: a measure of the development of shoots from buds at the base of the main stem. This can be expressed as a visual rating (on a scale of 1 to 9, with 1 being a high degree of tillering and 9 being no tillering. This can also be expressed as an actual number of tillers per plant.

Wild type: the normal condition, either with regard to a whole organism (wild-type strain), or with reference to a particular mutation (Birgid Schlindwein's Hypermedia Glossary of Genetic Terms).

### DETAILED DESCRIPTION OF THE INVENTION

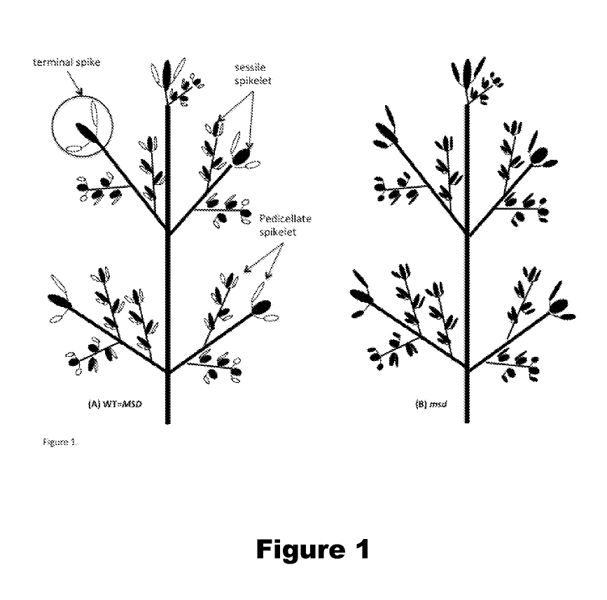

Normal sorghum lines produce a main inflorescence, primary branches, secondary, and tertiary branches, which all end with a terminal spike consisting of one sessile complete spikelet (fertile floret) and two sterile pedicellate spikelets (florets with a pedicel). One or more adjacent spikes may also develop below the terminal spike as shown in FIG. 1A. These adjacent spikes usually consist of one sessile and one pedicellate spikelet. In normal sorghum lines, only the sessile spikelets of terminal or adjacent spikes are complete flowers that will develop into mature seeds; the pedicellate spikelets are sterile and do not produce mature seed. In contrast, we have produced mutants or variants of sorghum which are associated with a phenotype wherein the pedicellate spikelets of the plant produce mature viable seed. These mutants, which are also referred to herein as sorghum multi-seeded mutants, msd, produce pedicellate spikelets which exhibit complete flowers with full development of functional gynoecium (ovary, style and stigma) and androecium (complete set of 3 anthers with copious amount of pollen). Seed yields per panicle in the msd mutants are greatly increased in comparison to normal wild-type sorghum, both in number of seeds produced and total seed weight per panicle or plant. This trait of the production of mature viable seed at the pedicellate spikelets in the msd mutants is stable and heritable. We have further discovered that the msd mutation is a monogenic recessive mutation, its heritability being consistent with a single recessive Mendelian trait. The msd mutants may be stably maintained by conventional intercrossing or selfing, or by tissue culture of regenerable cells.

The msd mutant sorghum lines of this invention are of the species Sorghum bicolor (L.) Moench, and apart from production of complete flowers and seed at the pedicellate spikelets (together with elongated pedicels, increased number of primary branches and length of primary and secondary branches), exhibit phenotypic traits typical of this species. Moreover, while these msd mutant sorghum produce mature viable seed at approximately all (defined as 95% or more) of the pedicellate spikelets when the plants are grown under non-stressed conditions, typically mature seeds are produced at all of the pedicellate spikelets under these conditions. As used herein, non-stressed conditions are defined as environmental conditions, including temperature and water, which over the course of the growing season from planting through harvest provide optimum growth of the sorghum plant. It is understood that actual non-stressed conditions will vary with the particular sorghum variety, soil conditions and geography, and may be readily determined by the skilled practitioner. Specifically, plant stress may be measured using techniques conventional in the art, such as measures of leaf water potential (Fisher D B, Cash-Clark C E, 2000, Gradients in water potential and turgor pressure along the translocation pathway during grain filling in normally watered and water-stressed wheat plants. Plant Physiol 123: 139-148) or elevated leaf temperatures (Leinonen I, Jones H G, 2004, Combining thermal and visible imagery for estimating canopy temperature and identifying plant stress. J Exp Bot 55: 1423-1431)].

Production of the msd mutant sorghum may be affected by treatment of seeds or regenerable cells (including tissue) of a starting parent or wild-type sorghum with physical or chemical mutagens, preferably chemical mutagens, under conditions effective to generate genetic mutations therein (mutagenesis). The particular sorghum line selected as the starting parent is not critical, although inbred, homozygous lines are preferred. A variety of mutagens are known in the art and are suitable for use herein, including but not limited to, X-ray and γ-irradiation, chemical mutagens such as ethyl methane sulfonate (EMS), methyl methane sulfonate (MMS), diethyl sulfate (DES), N-Nitroso methyl urea (NMU), and N-Nitroso ethyl urea (NEU), or combinations of chemical and irradiation mutagens. The methodology of inducing mutations in the sorghum seed or cells may be readily determined by the practitioner skilled in the art, and will of course vary with the specific mutagen selected. Conventional mutagenesis procedures which are suitable for use herein include those described by Quinby and Karper (1942. ibid), Sree Ramulu (1970a. ibid), Sree Ramulu (1970b. ibid), and Sree Ramulu and Sree Rangasamy (1972. ibid), the contents of each of which are incorporated by reference herein. Preferred mutagenesis procedures for use herein are described in Example 1.

The treated (mutagenized) seeds or treated cells are allowed to grow into plants, such as by planting or conventional tissue culture, respectively, producing an M₁ generation plant. These M₁ plants will not exhibit the multi-seeded phenotype because the mutation is monogenic recessive. Thus, the M₁ may then be selfed, and the resultant panicles harvested and planted to produce M₂ plants. M2 plants may be screened by visual observation to select for plants exhibiting the multi-seeded phenotype (pedicellate spikelets exhibiting complete flowers producing mature seed). In a preferred yet optional embodiment, the selected plants are selfed one or more times, with plants exhibiting the multi-seeded phenotype selected at each successive generation, to reduce other, undesired background mutations which may arise from the mutagenesis. In a particularly preferred embodiment, the selected mutant plants exhibiting the multi-seeded phenotype are backcrossed with the original non-mutated starting parent, and the backcross progeny selfed one or more times, wherein healthy plants of the F₂ and subsequent generation backcross progeny exhibiting the multi-seeded phenotype are selected and retained. Some M2 and M3 plants may exhibit undesired mutations, including production of some immature seed and partial male sterility. However, most of these background mutations are absent from M4 generation plants and are completely removed upon backcrossing with the original parent (non-mutated) parent sorghum. Moreover, when produced from inbred homozygous parents, the resultant backcrossed mutants exhibiting the multi-seeded phenotype will also be inbred homozygous.

A sample of at least 2,500 seeds of a preferred msd mutant sorghum line which was produced as described herein, referred to herein as msd1, and which may be used to produce hybrid sorghum exhibiting the multi-seeded phenotype, has been deposited under the conditions of the Budapest Treaty with the American Type Culture Collection (10801 University Blvd, Manassas, Va., 20110-2209, USA) on Aug. 3, 2012, and has been assigned deposit accession no. ATCC PTA-13113.

The deposited msd1 mutant sorghum line is an inbred homozygous line exhibiting the above-mentioned phenotype of pedicellate spikelets producing mature viable seed. The msd1 mutant line is also fully male fertile, producing viable pollen, and is characterized by the physiological and morphological characteristics shown in Table 1 as follows:

TABLE 1
Sorghum mutant line msd1 has the following characteristics based
on measurements collected at the USDA-ARS farm, Lubbock, TX.

| Agronomic | | | |
|---|---|---|---|
| Features | Traits | Mean value for msd1 | |
| A. Maturity | Days to flower | 52-58 | days |
| B. Plant | Height (cm) | 121.1 | cm |
| | Head exsertion | 3.3 | cm |
| | Plant Color | Red | |
| | No. of Tillers | 2-3 | |
| | Fertility reaction | B (maintainer of cytoplasmic | |
| | | male sterility) | |
| C. Leaf | Width | 8.2 | cm |
| | Length | 78.2 | cm |
| | No. per main stalk | 12 | |
| | Midrib color | White | |
| | Color and pattern | Solid dark green | |
| | Angle | 68-72° | |
| D. Panicle | Panicle Length | 33 | cm |
| | Panicle branch length (1°) | 9.8 | cm |
| | 1° Panicle branch | erect | |
| | orientation | | |
| | Panicle shape | Cylindrical | |
| | Glume color | Red | |
| | Awns | Absent | |
| | Head type | Semi open | |
| E. Kernel | Weight/100 seeds (g) | 1.9 | g |

| | |
|---|---|
| Seed size | 45,000-50,000 seeds/kg |
| Pericarp | Opaque |
| Testa | Absent |
| Endosperm Color | White |

The msd mutant sorghum plants described above may be crossed with other sorghum lines, particularly elite large-seeded lines, to generate fertile hybrids exhibiting the same multi-seeded phenotype and consequent improved grain yield (seed number and increased total seed weight). A variety of sorghum lines may be crossed with the msd mutants, including but not limited to KS115, described by Tuinstra et al. (2001. KS 115 Sorghum. Crop Sci. 41:932) and other desirable germplasm accessions available at the USDA, ARS, National Genetic Resources Program. Germplasm Resources Information Network—(GRIN), as PI 613536 (http://www.ars-grin.gov/cgi-bin/npgs/acc/display.pl?1600042). In producing a hybrid, first a sorghum plant is prepared from seed (or regenerable cells) of an msd mutant sorghum such as line msd1, described above, and crossed with a plant of a second sorghum variety to generate F₁ plants. The F₁ plants are selfed to generate subsequent generation (F₂) sorghum plants (hybrids), some of which will exhibit the multi-seeded phenotype wherein pedicellate spikelets produce viable seed, which are selected and recovered. These hybrid subsequent generation plants which exhibit a phenotype wherein pedicellate spikelets produce viable seed may be optionally further selfed, with progeny exhibiting the multi-seeded phenotype selected and retained.

General techniques for the production of sorghum hybrids are well-known and are described, for example, by Bading et al. (U.S. Pat. No. 8,212,126, the contents of which are incorporated by reference herein) and may involve the steps of: (1) planting in pollinating proximity seeds of a first and second parent sorghum plant (both parents being the msd mutant); (2) cultivating or growing the seeds of the first and second parent sorghum plants into plants that bear flowers; (3) emasculating the flowers of either the first or second parent sorghum plant, i.e. physically removing the anthers from the florets prior to blooming of the flowers so as to prevent pollen production or preventing dehiscence of pollen from anthers by introduction and maintenance of a high humidity environment by bagging a panicle or portion of a panicle with a plastic bag prior to blooming (a “wet pollination emasculation”) or by using as the female parent a male sterile plant, thereby providing an emasculated parent sorghum plant; (4) allowing natural cross-pollination to occur between the first and second parent sorghum plants or mechanically moving pollen from the pollen parent to the pollen sterile seed parent; (5) harvesting seeds produced on the emasculated parent sorghum plant; and, where desired, (6) growing the harvested seed into a sorghum plant, which may be a hybrid sorghum plant.

In an alternate embodiment of the invention, a tissue culture of regenerable cells of an msd mutant sorghum plant, including msd1, may be prepared. The regenerable cells in such tissue cultures may be derived from embryos, meristematic cells, microspores, pollen, anthers, stigma, flowers, leaves, stalks, roots, root tips, seeds, or from callus or protoplasts derived from those tissues. Techniques which are suitable for preparing and maintaining plant tissue cultures in this manner are well known in the art.

The following example is intended only to further illustrate the invention and is not intended to limit the scope of the invention that is defined by the claims.

### Example 1

### Materials and Methods

### Generation of the AIMS Mutant Library

The sorghum [Sorghum bicolor (L.) Moench] inbred line BT×623, which has served as a parent for several mapping populations and the source for genome sequencing [Paterson. 2008. Genomics of sorghum. International journal of plant genomics 2008:362451; Paterson et al. 2009. The Sorghum bicolor genome and the diversification of grasses. Nature 457:551-556); Bhattramakki et al. 2000. An integrated SSR and RFLP linkage map of Sorghum bicolor (L.) Moench. Genome 43:988-1002; Menz et al. 2002. A high-density genetic map of Sorghum bicolor (L.) Moench based on 2926 AFLP, RFLP and SSR markers. Plant molecular biology. 48:483-99; Subudhi and Nguyen. 2000. Linkage group alignment of sorghum RFLP maps using a RIL mapping population. Genome 43:240-9; Xu et al. 2001. Construction of genetic map in sorghum and fine mapping of the germination stimulant production gene response to Striga asiatica. Yi Chuan Xue Bao. 28:870-6], was used to generate the pedigreed mutant library. BT×623 seeds were obtained from the National Germplasm Resources Information Network of USDA-ARS (GRIN). Initial observations found that the seedlings from the original seeds showed minor variations in height and panicle size. However, no genetic heterogeneity was detected using 10 publicly available SSR markers [Menz et al. 2002. A high-density genetic map of Sorghum bicolor (L.) Moench based on 2926 AFLP, RFLP and SSR markers. Plant molecular biology. 48:483-99]. To ensure the homogeneity of the seeds used for mutagenesis, the original line was self-fertilized for six generations by single seed descent (SSD) to purify the line. At every generation, one plant that displayed the most typical characteristics of the original BT×623 was selected for further propagation. After six generations of selfing and purification, about 2 kg seeds were obtained for mutagenesis.

From the purified BT×623, batches of 100 g of dry seed (˜3300 seeds) were soaked with agitation (16 hours at 50 rpm on rotary shaker) in 200 ml of tap water containing EMS concentrations ranging from 0.1 to 0.3% (v/v). The treated seeds were subsequently thoroughly washed in about 400 ml of tap water for five hours at ambient temperature, changing the wash water every 30 min. Then the mutagenized seeds were air-dried and prepared for planting.

The air-dried seeds were planted at a density of 120,000 seeds per hectare. Before anthesis, each panicle was bagged with a 400 weight rain-proof paper pollination bag (Lawson Bags, Northfield, Ill.) to prevent cross pollination. After bagging, each bag was injected with 5 ml chlorpyrifos (Dow AgroSciences) at 0.5 ml/liter to control corn earworms that might hatch within the bag and destroy the seeds. Sorghum panicles were harvested manually and threshed individually. Each fertile panicle was planted as an M₂ head row. Three panicles from each M₂ head row were bagged before anthesis and only one fertile panicle was used to produce the M₃ seeds. Duplicate leaf samples were collected from the same fertile plant for extracting DNA, and both the leaf samples and the panicle were barcoded. To avoid cross-contamination of leaf samples with dead pollen that could fall onto the leaves during pollen shedding, leaves were thoroughly rinsed with de-ionized water before sampling. The seeds from the barcoded plants were harvested and used to propagate the M₃ generation. It should be noted that substantial mutant lines displayed diminished seed production during the M₃ generation. Thus, 10 panicles were bagged for each M₃ head row and pooled as M₄ seeds.

### Selection of msd Mutants

The msd mutants were screened by systematic and close inspection of panicles from each of the 6,144 M₃ plots from the beginning of grain filling to physiological maturity or harvesting. Any panicle with terminal spikes that developed into three seeds, instead of one seed and two aborted spikelets was selected and confirmed in the next generation. The confirmed mutants were backcrossed with the un-mutated BT×623 to reduce the background mutations.

### Characterization of msd Mutants

A total of 20 mutant lines were selected and used for confirmation and characterization (Table 3, note that mutant line msd1 is the same as msd-p12). Putative msd mutants were grown in 2 gallon pots in a greenhouse, with 4-5 seeds of each line were planted and eventually thinned out to 2 plants each pot. Plants were fertilized with Osmocote and irrigated regularly. At booting stage, samples of young panicles were obtained for microscopic examination and morphological characterization. At anthesis, spikelet samples were obtained from each line and observed for development of three seeds in the terminal spike. Histological observations were carried out using LEICA MZ6 microscope fitted with DFC420 camera. Photographs of developmental stages of spikelet development were obtained to investigate the nature of msd mutation effects on each line.

### Results and Discussion

From 6,144 independently mutated M3 lines, 20 msd mutants were selected over the past two years (Table 3). The first such mutant, msd1, was characterized in detail and was presented below.

Inflorescence Structure of msd1 Mutant

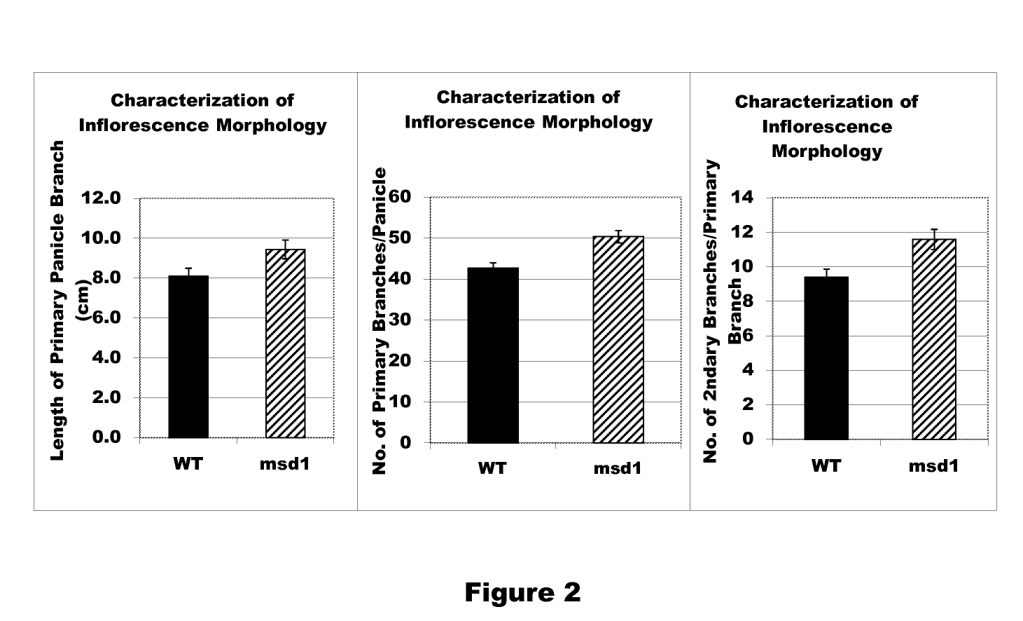

In sorghum, the development of reproductive structures in the pedicellate spikelets is suppressed at early development stages. In BT×623, the pedicellate spikelets only developed bracts and could only be seen from the adaxial (inside) side of an inflorescence branch but barely visible from the abaxial (outside) view. In msd1 mutants, the pedicellate spikelets were enlarged and could be seen clearly from both abaxial and adaxial views. In wild type BT×623, only the sessile spikelet developed into a seed. The pedicellate spikelets contained no gynoecium (ovary, style and stigma) or androecium (anther and filament) flower part and resulted in chaffy bracts. The pedicellate spikelets from the msd1 mutant produced perfect flowers and developed into seeds. To accommodate the increased number of fully developed flowers and seeds, the pedicels in msd1 mutant was much elongated. In addition to the altered fate of the pedicellate spikelets, the msd1 mutant also has distinct changes in inflorescence morphology. Those changes included increased number of primary branches and the length of primary and secondary branches (FIG. 2). Consequently, the msd1 panicle was more bulky and longer than the wild type BT×623.

Seed Production of msd1 Mutant

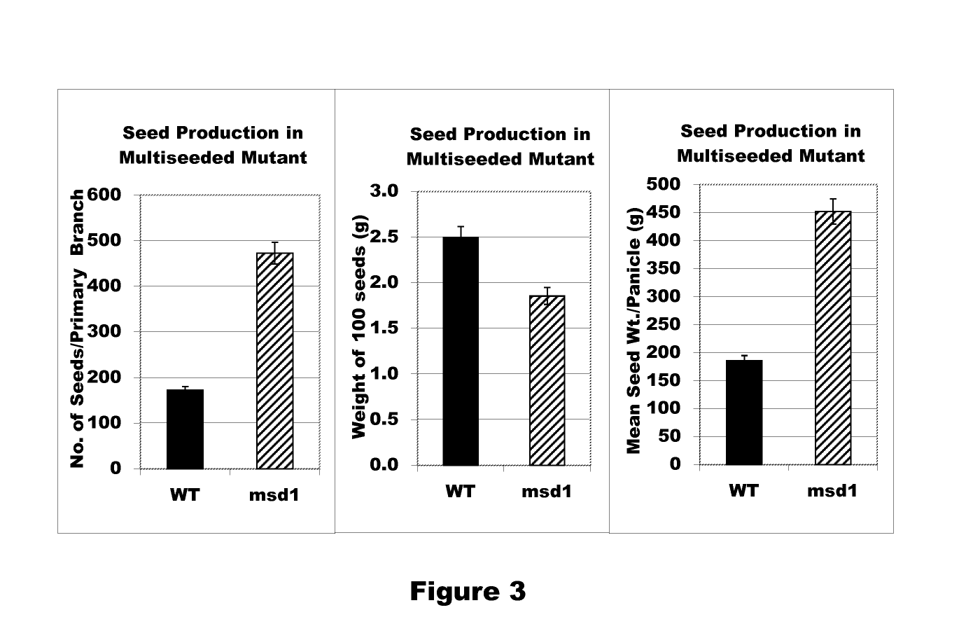

Due to the distinguished changes in inflorescence morphology and the fate of the pedicellate spikelets, the msd1 mutants approximately tripled the number of seeds produced per primary inflorescence branch that can be produced in wild type BT×623 (FIG. 3A). The increased seed numbers came with a cost of reduced seed size (FIG. 3B), which was more than compensated with increased seed number. Therefore, the seed production on basis of weight per panicle was more than doubled in msd1 mutant plants (FIG. 3C).

msd1 is a Monogenic Recessive Mutation

To determine the nature of the msd1 mutation, the msd1 mutant was crossed to the wild type BT×623. All F₁ plants had sterile and chaffy pedicellate spikelets and panicle morphology similar to BT×623, indicating the mutation is recessive. Among the 99 F₂ plants, 23 were msd1 mutants and 76 wild types (Table 2). This ratio is consistent with a single recessive Mendelian trait. Based on this experiment, the msd1 phenotype may be caused by a hypomorphic or amorphic mutation that blocked the function a single nuclear gene that serves as a repressor of the development of reproductive structures in the pedicellate spikelets in wild type sorghum. The most likely scenario is that in wild type sorghum, there is a pathway leading to the suppression of the pedicellate spikelets. Because it is unlikely to obtain a phenotype for hypomorphic mutation on redundant genes, some components of this pathway must be encoded by single nuclear gene. The msd mutants will serve as important tools to identify those components.

It is unclear how many loci are represented by the 20 msd mutants. Previously, we have isolated 21 confirmed brown midrib (bmr) mutants from 3000 M3 families. These 21 bmr mutants represented at least seven loci (Jeffrey Pedersen, personal communication). One bmr mutant is allelic to bmr2, four allelic to bmr12, six allelic to bmr6, six were novel bmr mutations representing 4 new loci [Saballos et al. 2012. Brown midrib2 (bmr2) encodes the major 4-coumarate:coenzyme A ligase involved in lignin biosynthesis in sorghum (Sorghum bicolor (L.) Moench). Plant J. 70:818-830; Sattler et al. 2012. Identification and Characterization of Four Missense Mutations in Brown midrib 12 (bmr12), the Caffeic O-Methyltranferase (COMT) of Sorghum. BioEnergy Research Available online: DOI 10.1007]. Based on the observations on bmr mutants, these 20 independent msd mutants most likely represent multiple loci.

In summary, we have isolated a series of msd mutants in sorghum. In general, these mutants display increased number and length of inflorescence branches, larger panicle size, and fully developed pedicellate spikelets. On weight basis, seed yield per panicle in msd1, the first characterized msd mutants, more than doubled that of the none-mutated inbred line BT×623. These mutants may have direct application as breeding materials to breed sorghum hybrids with high grain yield potential.

TABLE 2
Chi square analysis of BC₁F₂ generation
from msd12 backcross to WT.

| Phenotype | Observed | Expected | Chi-square | P value |
|---|---|---|---|---|
| WT = mono/single | 76 | 74 | 0.05 | |
| seeded (MSD) | | | | |
| Multi-seeded | 23 | 25 | 0.16 | |
| (msd) | | | | |
| Total | 99 | 99 | 0.21 (ns) | P = 0.41 |

TABLE 3
Morphological characterization of panicle characteristics
of wild type (MSD-P = single mono seeded) and multi-seeded
(msd) sorghum mutant lines grown in polyhouse.

| Mutant | | |
|---|---|---|
| Line ID | Pedigree | Observed panicle traits |
| msd1 | 20M2-0222 | 3-seeded terminal spikelets; multi- |
| (msd-p12) | | seeded; mature seeds developed in |
| | | sessile and pedicellate florets, robust |
| | | seed set. |
| msd-p1 | 10M2-0065 | 3-seeded terminal spikelets; multi- |
| | | seeded; mature and immature seeds |
| | | developed in sessile and pedicellate |
| | | florets, partial male sterility. |
| msd-p2 | 10M2-0181 | 3-seeded terminal spikelets; multi- |
| | | seeded; mature and immature seeds |
| | | developed in sessile and pedicellate |
| | | florets, very late flowering and |
| | | maturity, robust seed set. |
| msd-p3 | 10M2-0284 | 3-seeded terminal spikelets; multi- |
| | | seeded; mature and immature seeds |
| | | developed in sessile and pedicellate |
| | | florets, partial male sterility. |
| msd-p4 | 10M2-0358 | 3-seeded terminal spikelets; multi- |
| | | seeded; mature and immature seeds |
| | | developed in sessile and pedicellate |
| | | florets, partial male sterility. |
| msd-p5 | 10M2-0612 | 3-seeded terminal spikelets; multi- |
| | | seeded; mature and immature seeds |
| | | developed in sessile and pedicellate |
| | | florets, partial male sterility. |
| msd-p6 | 10M2-0640 | 3-seeded terminal spikelets; multi- |
| | | seeded; mature and immature seeds |
| | | developed in sessile and pedicellate |
| | | florets, partial male sterility. |
| msd-p7 | 10M2-1448 | 3-seeded terminal spikelets; multi- |
| | | seeded; mature and immature seeds |
| | | developed in sessile and pedicellate |
| | | florets, partial male sterility. |
| msd-p8 | 10M2-1676 | 3-seeded terminal spikelets; multi- |
| | | seeded; mature and immature seeds |
| | | developed in sessile and pedicellate |
| | | florets, partial male sterility. |
| msd-p9 | 15M2-1344 | 3-seeded terminal spikelets; multi- |
| | | seeded; mature and immature seeds |
| | | developed in sessile and pedicellate |
| | | florets, partial male sterility. |
| msd-p10 | 15M2-1408 | 3-seeded terminal spikelets; multi- |
| | | seeded; mature and immature seeds |
| | | developed in sessile and pedicellate |
| | | florets, partial male sterility. |
| msd-p11 | 20M2-0146 | 3-seeded terminal spikelets; multi- |
| | | seeded; mature and immature seeds |
| | | developed in sessile and pedicellate |
| | | florets, partial male sterility. |
| msd-p12 | 20M2-0222 | 3-seeded terminal spikelets; multi- |
| | | seeded; mature seeds developed in |
| | | sessile and pedicellate florets, robust |
| | | seed set. |
| msd-p13 | 20M2-0736 | 3-seeded terminal spikelets; multi- |
| | | seeded; mature and immature seeds |
| | | developed in sessile and pedicellate |
| | | florets, partial male sterility. |

It is understood that the foregoing detailed description is given merely by way of illustration and that modifications and variations may be made therein without departing from the spirit and scope of the invention.

We claim:

1. A sorghum (Sorghum bicolor L. Moench) plant or parts thereof comprising a phenotype wherein pedicellate spikelets produce mature viable seed.

2. The sorghum plant or parts thereof of claim 1 comprising a phenotype wherein approximately all sessile and pedicellate spikelets produce mature viable seed when grown under non-stressed conditions.

3. The sorghum plant or parts thereof of claim 1 comprising a phenotype wherein all sessile and pedicellate spikelets produce mature viable seed when grown under non-stressed conditions.

4. The sorghum plant or parts thereof of claim 1 selected from the group consisting of msd1, representative sample of seed thereof having been deposited as ATCC deposit accession number PTA-13113, and progeny thereof.

5. The sorghum plant or parts thereof of claim 1 which is fully male fertile.

6. The sorghum plant or parts thereof of claim 2 which is fully male fertile.

7. The sorghum plant or parts thereof of claim 1 which is inbred, homozygous.

8. The sorghum plant or parts thereof of claim 2 which is inbred, homozygous.

9. Seed of said sorghum plant or parts thereof of claim claim 1.

10. An ovule of said sorghum plant or parts thereof of claim 1.

11. Pollen of said sorghum plant or parts thereof of claim 1.

12. A tissue culture of said sorghum plant or parts thereof of claim 1.

13. A seed of sorghum plant line msd1, representative sample of said seed thereof having been deposited as ATCC deposit accession number PTA-13113.

14. A sorghum plant or part thereof produced from said seed of claim 13.

15. The sorghum plant of claim 14 produced by growing said seed.

16. A sorghum plant produced by crossing said sorghum plant of claim 1 comprising a phenotype wherein pedicellate spikelets produce viable seed, with a second sorghum plant.

17. The sorghum plant of claim 14 wherein said second sorghum plant is different from said sorghum plant comprising a phenotype wherein pedicellate spikelets produce viable seed.

18. A method for producing hybrid sorghum comprising a phenotype wherein pedicellate spikelets produce viable seed comprising:
a) preparing a first sorghum plant from seed of sorghum plant line msd1, representative sample of said seed having been deposited as ATCC deposit accession number PTA-13113;
b) crossing said first sorghum plant with a plant of a second sorghum variety to generate F₁ sorghum plants,
c) crossing said F₁ plants as both the male and female parent to generate a subsequent generation sorghum plant, wherein some of said subsequent generation sorghum plants will exhibit a phenotype wherein pedicellate spikelets produce viable seed; and
d) recovering said subsequent sorghum plants which exhibit a phenotype wherein pedicellate spikelets produce viable seed.

19. A mutant sorghum plant comprising a phenotype wherein pedicellate spikelets produce mature viable seed, produced by the process comprising:
a) exposing plants or parts thereof of an inbred, homozygous parent sorghum line to a mutagen under conditions effective to generate genetic mutations therein;
b) growing the mutagen-exposed plants or parts thereof to produce mature flowering plants, self-pollinating said mature flowering plants, and recovering and planting seed therefrom to produce a first subsequent generation;
c) recovering and planting seed from plants of said subsequent generation possessing panicles with pedicellate spikelets which produce mature viable seed to produce putative multi-seeded mutants;
d) backcrossing said putative mutants with said parent sorghum line and selecting and recovering mutant sorghum plants comprising a phenotype wherein pedicellate spikelets produce mature viable seed.
